(19) United States
(12) Patent Application Publication
(10) Pub. No.: US 2026/0301919 A1
(43) Pub. Date: Oct. 1, 2026

(54) PATIENT CARE SYSTEM

(71) Applicant: SANOFI (CHINA) INVESTMENT CO., LTD., Beijing (CN)

(72) Inventors: Marie Chavanon, Shanghai (CN); Lei Yang, Shanghai (CN)

(21) Appl. No.: 19/490,255

(22) PCT Filed: Jun. 5, 2024
(86) PCT No.: PCT/CN2024/097589

§ 371 Date: Dec. 4, 2025

(30) Foreign Application Priority Data
Jun. 5, 2023 (CN) 202310659795.6

(51) Int. Cl. G16H 20/70

(52) U.S. Cl. CPC G16H 20/70

(57) ABSTRACT

A patient care system provides intervention courses with patients having a physical health condition, and said system comprises a computing device, and a storage unit in which a list of one or more application programs is stored, said programs can be executed for providing instructions for a plurality of steps of purpose identification, condition assessment, mapping, and intervention training. The system could improve quality of life, alleviate the severity of the condition and reduce relapse frequency.

### TECHNICAL FIELD

The present invention relates to a patient care system for providing intervention courses to patients having a physical health condition.

### BACKGROUND

A physical health condition, such as cancer, asthma, or other dermatology or inflammatory diseases (e.g. atopic dermatitis, also called eczema, Chronic obstructive pulmonary disease, Bullous Pemphigoid and Chronic Spontaneous Urticaria), could lead to other health conditions including symptoms and concomitant diseases, such as food allergies, hay fever, and those dietary and physical exercise related conditions, especially those mental health related conditions, such as insomnia, anxiety, depression, fatigue, etc. Mental health problems could bring lots of troubles, such as non-compliance of prescription, prolonged course of disease, etc.

Current treatment processes for patients having a physical health condition focus on medication and daily care on body part, but provide less help on mental health of patients. There are unmet needs for patients having a physical health condition, to perform regularly monitoring, assessment, and intervention on mental health in a convenient way.

### SUMMARY OF THE INVENTION

An object of the invention is to provide a patient care solution that provides cognitive and behavioral training courses to patients having a physical health condition to improve quality of life, alleviate severity of the condition and reduce relapse frequency.

According to one aspect of the present invention, the present invention provides a patient care process for providing intervention courses to a patient having a physical health condition, including the steps of
retrieving a list of a plurality of health conditions caused by the physical health condition; providing said plurality of health conditions to the patient;
receiving an input from the patient, that specifies one health condition selected from said plurality of health conditions by the patient;
providing a group of questions corresponding to said one health condition to the patient to obtain feedback to the group of questions; and
selecting one or more intervention courses based on the feedback and providing said one ore more intervention courses to the patient.

In the disclosed embodiments, said process further comprises a step of receiving an identification of one or more physical health conditions of the patient.

In the disclosed embodiments, said one health condition is selected in a first round selection and a second round selection, and
in the first round selection, more than one health conditions are selected from said plurality of health conditions by the patient; and
in the second round selection, said one health condition is selected from said more than one health conditions by the patient.

In the disclosed embodiments, the list of said plurality of health conditions is essentially consisting of mental health conditions.

In the disclosed embodiments, the list of said plurality of health conditions comprises fatigue, insomnia, frustration, anxiety including fear of going out, appearance anxiety, uncertainty about recovery time, uncertainty about cause of disease, uncertainty about diet, fear of colleagues or classmates' views, worry about drug dependence, worry about drug side effect, worry about the disease not being completely cured, negative impact on social relationships, unable to concentrate on work or study, worry about medical expenses and heavy financial burden, fear of doing wrong physical exercise, fear of needle injection and/or anxiety over food and other allergens.

In the disclosed embodiments, said plurality of health conditions comprises pain.

In the disclosed embodiments, the plurality of health conditions are classified into condition groups comprising the groups of sleep, anxiety, and depression.

In the disclosed embodiments, the group of questions is designed to evaluate a scale corresponding to said one health condition, and said one or more intervention courses are provided base on the calculated score of said scale.

In the disclosed embodiments, the one or more intervention courses relate to mental health.

In the disclosed embodiments, said one or more intervention courses can be classified into different levels comprising a level of relaxation tools and a level of cognitive behavioral treatments depending on the severity of said one health condition indicated by the calculated score.

In the disclosed embodiments, the one or more intervention courses comprises cognitive part and behavior part.

In the disclosed embodiments, the physical health condition is cancer, asthma, atopic dermatitis, chronic obstructive pulmonary disease, bullous pemphigoid or chronic spontaneous urticaria.

Another aspect of the present invention is to provide a patient care system for providing intervention courses to patients having a physical health condition, said system comprising
a computing device which comprises an input interface and an output interface, and
a storage unit in which one or more application programs is stored, said one or more programs can be executed for providing instructions for a plurality of steps comprising
retrieving a list of a plurality of health conditions caused by said physical health condition;
providing the list of said plurality of health conditions through the output interface of the computing device;
in response to receiving a first input through the input interface of the computing device, that specifies one health condition, providing a group of questions corresponding to said one health condition through the output interface of the computing device; and
in response to receiving a second input through the input interface of the computing device, that comprises feedback to said group of questions, selecting one or more intervention courses based on said feedback to said group of questions, and providing one or more intervention courses through the output interface of the computing device.

In the disclosed embodiments, said steps further comprise identifying one or more physical health conditions of the patient.

In the disclosed embodiments, said plurality of steps further comprising
in response to receiving an input through the input interface of the computing device, that specifies one or more health conditions of said plurality of health conditions, providing an output through the output interface of the computing device, that requires that said one health condition of said one or more health conditions is specified; and
and receiving said first input through the input interface of the computing device.

In the disclosed embodiments, the list of said plurality of health conditions is essentially consisting of mental health conditions.

In the disclosed embodiments, the list of said plurality of health conditions comprises mental health conditions, dietary related conditions and/or physical exercise related conditions.

In the disclosed embodiments, the list of said plurality of health conditions comprises fatigue, insomnia, frustration, anxiety and/or pain.

In the disclosed embodiments, said group of questions is designed to evaluate a scale corresponding to said one health condition, and said one or more intervention courses are provided depending on a scale score calculated based on the completed group of questions.

In the disclosed embodiments, said one or more intervention courses relate to mental health, dietary behavior or physical exercise behavior.

In the disclosed embodiments, wherein said one or more intervention courses can be classified into different levels comprising a level of relaxation tools and a level of cognitive behavioral treatments depending on the severity of the health condition indicated by the calculated score.

In the disclosed embodiments, said one or more intervention courses comprises cognitive part and behavior part.

In the disclosed embodiments, said storage unit is a part of said computing device.

In the disclosed embodiments, said input interface comprises a display and a graphical user interface.

In the disclosed embodiments, said output interface comprises a display and a graphical user interface.

In the disclosed embodiments, said input interface and said output interface comprise a same display and a same graphical user interface.

In the disclosed embodiments, said graphical user interface comprises a main page, a treatment page, a recording page, an explore page, and a personal profile page.

In the disclosed embodiments, said system further comprises a processor which calculate said score of the scale based on the feedback.

In the disclosed embodiments, said processor is a part of said computing device.

In the disclosed embodiments, said system further comprises an administrative unit which could monitor and modify said application programs, said plurality of health conditions, and said group of questions.

In the disclosed embodiments, said system further comprises a communication interface which could connect the computing device to a network or a Cloud.

In the disclosed embodiments, said application programs can be stored in the storage unit of the network or Cloud.

In the disclosed embodiments, said one or more application programs are installed on a third party platform.

In the disclosed embodiments, said Cloud or network comprises a processor and said processor could calculate the scale score based on the feedback to the group of questions.

In the disclosed embodiments, said group of questions and the list of said plurality of health conditions are stored on said Cloud or network.

In the disclosed embodiments, said one or more application programs are WeChat mini-programs.

In the disclosed embodiments, said one or more application programs are mobile applications installed under iOS or Android environments.

In the disclosed embodiments, said computing device is a smart phone, a computer tablet, or a PC.

In the disclosed embodiments, the physical health condition is cancer, asthma, atopic dermatitis, chronic obstructive pulmonary disease, bullous pemphigoid or chronic spontaneous urticaria.

Another aspect of the present invention is to provide a method for providing intervention courses to a patient having a physical health condition, said method comprising
providing the computing system according to the disclosed embodiments of the present invention, and
executing said one or more programs for providing instructions for said plurality of steps.

The invention is not limited thereof and can be applied to improve patient's life and condition in various disease areas including but not limited to oncology, dermatology, inflammatory diseases, etc. Other features and advantages of the present invention will become apparent from the reading of the following detailed description made with reference to the appended drawing.

### BRIEF DESCRIPTION OF DRAWINGS

FIG. 1 is a flow chat of the present inventive process according to an embodiment of the invention.

FIG. 2 is a block diagram of the patient care system according to an embodiment of the invention.

### DETAILED DESCRIPTION OF EXEMPLARY EMBODIMENTS

The present invention relates to an evidence-based, personalized psychological intervention solution for patients having a physical health condition that improves or alleviates the health conditions resulted from the physical health condition and eventually reduces the severity, and frequency of relapse of the physical health condition and improve quality of life of the patients. The present invention is not limited thereof and can be applied in various disease areas including oncology, dermatology and inflammatory diseases.

A physical health condition is defined by opposition to a mental health condition. It may mean any illness or dysfunction of, or injury to, the human body. In relative to a physical health condition, a mental health condition means any psychiatric condition that that affects a person's thinking, feeling, behavior or mood and may impair a personal's judgement or emotional stability. In some embodiments, a physical health condition is a primary health condition suffered by a patient. A list of health conditions are secondary conditions resulted from the primary health condition, including symptoms and concomitant conditions thereof, especially those mental health related conditions.

In some embodiment, the present invention provides a process for providing intervention courses to patients having a physical health condition, which comprises steps of purpose identification, assessment, mapping, and training.

When intervention courses are targeted to one physical health condition, it may be necessary to select such one condition from multiple physical health conditions at the beginning. Such a physical health condition could be selected as the primary health condition, such as by an input from patients. In the purpose identification (also called selection) step, related health conditions, such as mental health conditions, could be selected by a patient having the primary health condition, from a list of the health conditions which could be resulted from such primary health condition. Such selected health conditions are conditions which the patient is suffering from and expects to be solved. In some embodiments, the selections are run for more than one round, preferably two rounds. In the first-round selection, a user, preferably a patient, is allowed to select one or more conditions. In the second-round selection, the user is required to select only one condition among the conditions selected in the first round. Under such selection design, a patient identifies one condition the patient thinks most urgently needs to be solved and the intervention purpose is determined. Such arrangement makes the patient believe that the patient controls the selection process and the last choice comes from within the patient self. Such an auto-suggestion process can result in positive impact on the patient and make it easier for the patient to follow the intervention courses and the guidance delivered by the courses later.

In some embodiments, the list of the health conditions could be stored in a condition database before the selection. The condition database could be set up at any appropriate location, such as a user's device, say a patient device, an HCP (Healthcare Professional) device, or an administrator's device, a server, or a Cloud. These conditions are related to a physical health condition and could be collected from and updated in any appropriate ways, such as investigations, surveys, or research on patients, HCPs, journals, handbooks, etc. In some embodiments, the database comprises 10-30 conditions. Preferably, the conditions are essentially mental health related. In some embodiments, the number of the mental health conditions is more than 80%, preferably more than 85%, more preferably more than 90% of the number of the total conditions.

In some embodiments, a list of health conditions comprises fatigue, insomnia, frustration, anxiety, such as fear of going out, appearance anxiety, uncertainty about recovery time, uncertainty about cause of disease, uncertainty about diet, fear of colleagues or classmates' views, worry about drug dependence, worry about drug side effect, worry about the disease not being completely cured, negative impact on social relationships, unable to concentrate on work or study, worry about medical expenses and heavy financial burden, fear of doing wrong physical exercise, fear of needle injection, and anxiety over food and other allergens. In some embodiments, the list of conditions further comprises pain.

Preferably the list of conditions does not comprise any condition exhibited by nearly all patients who have the same physical health condition. For example, in atopic dermatitis patients, the list of conditions preferably does not comprise the condition itching. The condition itching occurs in nearly all atopic dermatitis patients and is the top concerns to most patients. If itching is included in the conditions list, most patients would select it. Under such situation, it is difficult for the cognitive and behavioral training (CBT) programs to differentiate patients' condition status and to give intervention courses suitable to each patient.

The conditions to be selected by a user in the intervention training process according to the present invention are retrieved by an instruction of an application program in response to an operation of a user, such as a patient. The user could select one or more conditions therefrom, which are thought to be currently relevant to the patient. Normally an adult patient self could operate and complete the selection. For young children, adults, normally their parents, operate and complete the selections for them.

When a final choice is identified, a group of questions is provided with the patient for condition assessment. The patient is required to provide feedback to the group of questions. The group of questions includes questions or inquiries corresponding to the finally selected condition so that an assessment on the patient current status, such as the severity of the condition, could be conducted based on the patient's feedback to the group of questions. One or more groups of questions are set up for evaluating various conditions. In some embodiments, the groups of questions are questionnaires known in the art and used as criteria in clinical diagnosis. A questionnaire is normally designed so that feedback to the questionnaire received from a patient allows calculating a severity score on a scale corresponding to the health condition of the patient.

In some embodiments, the questionnaires are classified into groups depending on various condition groups. For example, one questionnaire could be provided corresponding to one or more conditions and such one or more conditions can be a condition group. One or more questionnaires could be provided in response to one condition and such one or more questionnaires can be a questionnaire group. In some embodiments, corresponding to the condition “pain”, a questionnaire group is designed to evaluate scales, such as a group including DLQI (Dermatology Life Quality Index). In some embodiments, corresponding to a condition group of insomnia and fatigue, a questionnaire group is designed to evaluate the scale, such as a group including PSQI (Pittsburgh Sleep Quality Index). In some embodiments, corresponding to the condition depression, a questionnaire group is designed to evaluate the scale, such as a group including PHQ-9 (Patient Health Questionnaire). In some embodiments, corresponding to a condition group of various anxieties, a questionnaire group is designed to evaluate the scale, such as a group including STAI (State-Trait Anxiety Inventory). Preferably, the patient is required to complete all questions or inquiries in the groups of questions. But in some embodiments, it is sufficient for meeting the purpose of the present invention to evaluate the status of the health condition even based on partially completed groups of questions.

After the user completes the group of questions, an assessment is conducted. In some embodiments where the groups of questions are designed to evaluate specific scales, scale scores can be calculated based on the answer to each question of the questionnaire. In some embodiments, such calculation is automatically performed by an application program.

The assessment results reflect condition status of the patient, such as the severity of the condition, and could map to intervention courses. Depending on various outcomes of the group of questions, such as scores calculated based on completed questionnaire, intervention courses are selected and provided to the patient.

In some embodiments, the intervention courses aim at bringing positive changes in patient's cognitive and behavioral aspects. Such treatment process can be called a cognitive and behavioral training (CBT) program. Regarding the cognitive aspect, the courses may relate to sleep hygiene, cognitive reconstruction including cognitive distortion and cognitive correction. Regarding the behavioral aspect, the courses may relate to sleep diary, relaxation, food diary, and emotional diary. The courses aiming at impacting the behavior could also include cognitive content. For example, why relaxation is needed and what a sleep diary could be. The cognitive-related courses could also include behavior-related content. For example, after acknowledging sleep hygiene, it is expected that the patient follows the guidance upon sleeping. Children normally need a special intervention plan, which not only changes the cognitive of children, but also the cognitive of their parents. So, the parents are also recommended to receive such intervention courses. Normally, teenagers and adult can use a similar plan.

In some embodiments, the courses corresponding to an insomnia patient include sleep limitation, stimulation limitation, relaxation training, sleep hygiene, and cognitive reconstruction.

In some embodiments, depending on the scores calculated for the scale, such as STAI, the condition anxiety could be classified into anxiety personality and instant anxiety. For anxiety personality, the courses could include relaxation training, identification and evaluation of function disorder belief and intervention in automatic thinking; for instant anxiety, the courses could include tips on handling normal condition problems, humanistic care, daily condition management, condition medicament cognizing, and conditions cognizing.

In some embodiments, regarding the condition depression, depending on the scale score, the patient could be classified into anxiety group or insomnia group, and related intervention courses would be respectively provided.

In some embodiments, regarding the condition pain or another health condition resulting from a dermatological disease, depending on the score calculated for the scale such as DLQI, the courses include tips on handling such health conditions. For example, these could include tips on handling normal disease problems, daily disease management, disease medicament cognizing, and disease cognizing.

The intervention courses could be in various forms, such as animation, games, video, audio, text, etc.

In some embodiments, the intervention courses could be provided at different levels depending on the severity of the health condition indicated by the calculated scores. For example, for level 1 which represents a relative mild condition, courses comprising relaxation tools, say music for relaxation, are provided; for level 2 which represents a relative serious condition, courses comprising cognitive behavioral treatment programs are provided.

The intervention courses could improve or alleviate the severity of the corresponding secondary health condition, i.e. in some embodiments the mental health condition, and eventually bring benefits to the primary health condition, i.e. in some embodiments the physical health condition.

The intervention process could be conducted on a regular basis, such as weekly or fortnightly. In some embodiment, a treatment cycle of the intervention process could be 1-4 weeks. An HCP could then be involved to evaluate the result. If necessary, another treatment cycle could be performed. Normally, it is expected that the selected condition could be alleviated within 3 treatment cycles.

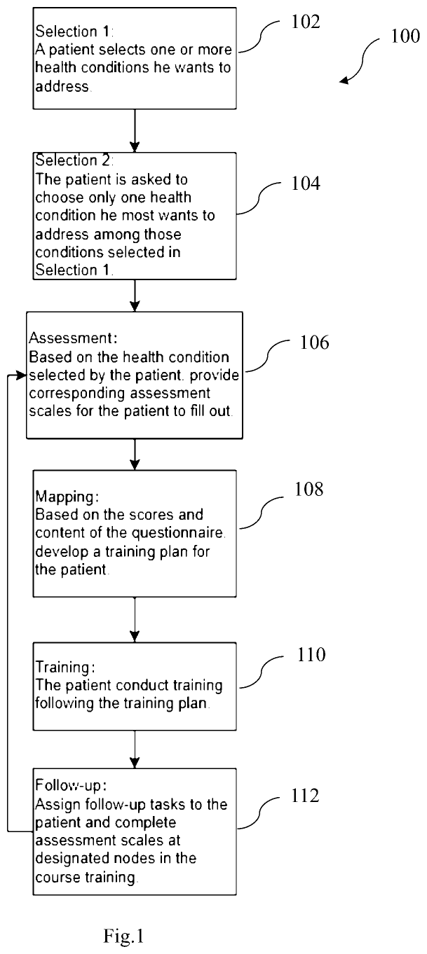

FIG. 1 is a flow chart illustrating Process 100 in accordance with some embodiments of the present invention. The step of purpose identification includes two rounds of selection. In Selection 1 (Step 102), a patient selects one or more health conditions the patient wants to address. In Selection 2 (Step 104), the patient is asked to choose only one health condition mostly wanted to address among those conditions selected in Selection 1. After the two selections, in the next step of Assessment (Step 106), based on the health condition selected by the patient as above, corresponding assessment scales are provided to the patient to fill out. Then, in the next Mapping step (Step 108), the scores are calculated based on the completed scales and a training plan is developed based on the calculated scores and other contents of the completed questionnaire. Next, in the Training step (Step 110), the patient conducts the training following the training plan. In the illustrated process, one more step is added. As a “Follow-up” step (Step 112), a follow-up task, such as additional assessments, is provided in designed nodes in the course training, such as once every two weeks. The patient is required to go back to Step 106 to complete the assessment task, so that the training plan could be adjusted accordingly based on the most updated outcome from the completed task.

The present invention also provides a patient care system for providing intervention courses with patients having a physical health condition. The system comprises a computing device and a storage unit. The computing device comprises at least one input interface and at least one output interface. One or more application programs could be stored on the storage unit.

In some embodiments, the application programs are configured for execution by a computing system having a processor to provide instructions for a plurality of steps comprising purpose identification, assessment, mapping, intervention training as above. The application programs could be applications installed under a computing environment, such as iOS, Android, macOS, or Microsoft Windows, and users could have access to the system through a computing device. The computing device is a device which could perform some or all functions of a computer, comprising a smart phone, a computer tablet or a PC (personal computer). The application programs could be stored in the storage unit of the computing device, and could be retrieved and executed by a processor to provide instructions. The application programs could be set up at a third party platform, such as a WeChat mini-program. The application programs could be available to users at various app stores (such as mobile application stores), third party platforms, or any location assigned by an administrator, or could be pre-installed in a computing device.

In some embodiments, the patient care system could comprise a computing device at user end. The user could be a patient, a HCP, or a person providing maintenance service to the system, such as an administrator. The computing device comprises at least one input interface and at least one output interface for information and data exchanging with users and the other parts of the system.

In the step of purpose identification, a list of health conditions is retrieved from a condition database and provided to the output interface of the computing device. A user conducts the operation of selection on the computing device. In some embodiments, the user could select one or more conditions in the first round of selection. The selection outcome could be received by the input interface of the device. In response to the selection outcome, the application program gives further instruction through the output interface of the device, requiring the user to conduct the second round selection to identify the final selection. The final selection is one condition which the user thinks most urgent to improve or alleviate. In the step of assessment, the final selection is provided with the system through the input interface of the device. Based on the final selection, the application program gives instructions to retrieve a group of questions to provide to the user through output interface of the device. The user completes the group of questions and the result is provided to the system through the input interface of the device. In the mapping step, the computing program retrieves intervention courses corresponding to the assessment result. In the step of intervention, the courses are provided to the user through the output interface of the device, and the user then performs the intervention courses.

The computing device comprises one or more storage units. The storage unit could be one or more memories including high-speed random memory, such as DRAM, SRAM, DDR RAM, or other random-access solid-state memory devices, and could be one or more non-volatile memories, such as magnetic disk storage devices, optical disk storage devices, flash memory devices, or other non-volatile solid-state storage devices. The application programs could be stored on the storage unit and are readable and executable to the computing device.

In some embodiments, the lists of conditions and intervention courses could be stored in database modules as long as the computing device could retrieve them. In some embodiments, such database modules and the storage unit for storing application programs are parts of the computing device and therefore they could be located within the computing device. In some embodiments, the lists of conditions and intervention courses, and application programs are directly stored on the storage unit of the computing device. In some embodiments, the storage unit includes one or more storage devices remotely located from the computing device, such as located in a separate computing device, an intranet, internet or a Cloud.

In some embodiments, the system further comprises a processor which could execute the application programs and generate instructions in each step. In some embodiments, such processor is a part of the computing device and therefore it could be located within the computing device. In some embodiments, the function of such processor could be provided by a network or a Cloud, and in some embodiments, such processor is a part of a network and a Cloud.

In some embodiments, the input interface of the computing device could be a manual input device, such as a computer mouse, a keyboard, or a touch screen. A user, such as a patient, could use the input device to manually provide inputs to the system, such as to complete the condition selections and assessments. Preferably, the input interface is the touch screen of a mobile phone or a computer tablet. Alternatively, the input interface could comprise a microphone and voice recognition hardware and software.

The output interface could be a screen (display). In some embodiments, the input interface and the output interface could be a same touch screen.

In some embodiments, the input interface and the output interface could comprise a graphic user interface. Preferably, the input interface and the output interface could comprise a same graphic user interface. The graphic user interface could be in the form suitable for a computing system and the user device. For example, such graphical user interface comprises a main page, a treatment page, a recording page, an explore page, and a personal profile page. In the main page, a list of pending tasks could be presented, as well as virtual buttons for directing the user to other pages. Recommended intervention course could be included in the treatment page. The user could follow directions to complete the intervention courses here. In the recording page, the user could complete emotional diary, conduct assessments on sleep, emotion, diet, and other aspects, and check the feedback of completed assessments. In the explore page, the user could learn the condition related knowledges in various forms, such as videos, articles, etc. In the personal profile page, the user could input personal information and check personal archives, health reports, training record, etc.

In some embodiments, the system could further comprise an administrative unit, through which a user (normally an administrator) could have an administrative right to monitor the operation, and the input and output of records and data, and could modify or update the application programs, the list of health conditions, and the group of questions. The administrative unit could comprise a user interface configured for administration purpose.

In some embodiments, the system further comprises a communication interface which includes communication parts and could connect the computing device to a network, say internet or intranet, or a Cloud. The system could make use of a plurality of physical and/or virtualized servers in the network or Cloud to store the groups of questions, the list of conditions, intervention courses, application programs, as well as generated data. The computing device could retrieve or has access to the data, information and application programs stored in the network and Cloud. For example, when the computing device is a mobile phone, the communication interface comprises parts used for mobile cellular systems of GRPR (general packet radio service), UMTS (Universal Mobile Telecommunications System) or others.

When the patient care system is set up at a Cloud, the storage of the list of conditions and the group of questions, and the processor function could use Cloud services. Under a system tech architecture of a patient care system set up in a Cloud in accordance with some embodiments of the present invention, a “Users” block represents devices at user end, including Patient Application, WeChat mini-program, Doctor Application, Management Center (such as an administrator device). In some embodiments, the devices at user end include both the input interface and output interface, which are used for users to provide inputs to the patient care system and also show outputs from the system to users. A “Cloud Network Facilities” block provides entrance and firewall connecting to the Cloud, including Cloud Gateway, Cloud Firewall, etc. A “Cloud CDN” could work as the export facilities of the Cloud network. A “Cloud Service” block provides the Cloud services including Cloud CLB, TKE K8s, instant message, SMS services, etc. “Production Environment” provides both storage and calculation functions, including Cloud TDSQL MySQL, Cloud Redis, Cloud DBS, etc. In some embodiment, Cloud TDSQL MySQL including both Primary and Slave provides database functions, and the group of questions and the condition lists could be stored therein. “Production Environment” could also store training courses in the forms of video, audio and pictures. The Cloud also acts as a platform for Security Services including Cloud Workload Protection, Data Security Audit, Vulnerability Scan Services, etc.

The present invention brings cognitive and behavioral treatment programs to patients having a physical health condition. Based on the auto-suggestion principle, thanks to the special design of the present invention, the multi-selection scheme, a patient could identify the condition most urgent to be solved. In some embodiments, such condition is a mental health condition resulted from the physical health condition. Such a way of identification could strengthen the patient's belief of controlling the selection processes and further make the patient easier to follow and complete the intervention courses, and to be in compliance with the guidance and knowledge obtained from the training. The inventive intervention process could improve or alleviate the severity of the selected condition, and eventually be advantageous to the recovery of the physical health condition and could improve the quality of life of patients.

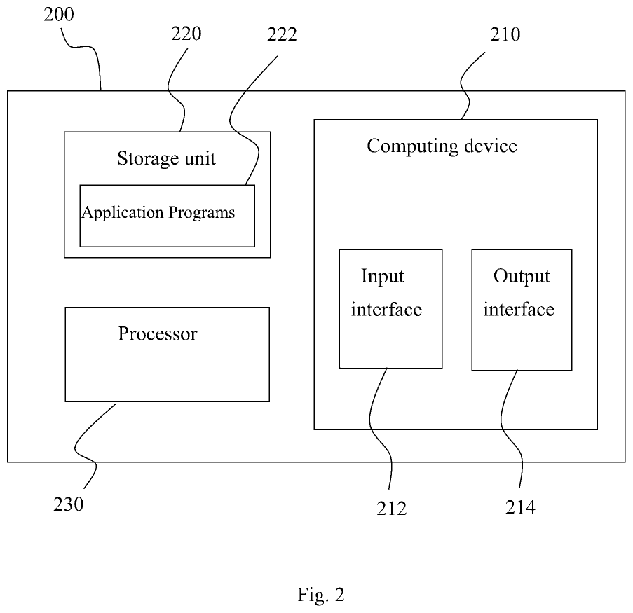

FIG. 2 shows a schematic example of the patient care system. It will be appreciated by the skilled person that other types of the system may be alternatively designed and used for the purpose of the present invention. The system 200 comprises a computing device 210, and a storage unit 220, wherein the computing device 210 further comprises an input interface 212 and an output interface 214. The system 200 provides outputs through the output interface 214, say to patients, and receives inputs through the input interface 212, say from patients.

The storage unit 220 comprises one or more storage devices, such as volatile memory or non-volatile memory. The storage unit 220 could be a part of the computing device 210, but could also be located in a separate device remotely from the computing device 210 as described above. The storage unit 220 stores one or more application programs 222. The application program 222 is in computer readable and executable form.

The system 200 could further comprises one or more processors 230. The processor 230 may be a general purpose processor and could be a single core device or a multiple core device. The processor 230 may comprises a central processing unit (CPU) or a graphical processing unit (GPU). Alternatively, the processor 230 may comprise specialized processing hardware, for instance a RISC processor or programmable hardware with embedded firmware. The processor 230 could access and execute the application program 222 so that operation instructions could be generated. The processor 230 could be a part of the computing device 210, but could also be located in a separate device remotely from the computing device 210 as described above.

While at least one exemplary embodiment has been presented in the foregoing detailed description, it should be appreciated that a vast number of variations exist. It should also be appreciated that the exemplary embodiment or embodiments described herein are not intended to limit the scope, applicability, or configuration of the claimed subject matter in any way. Rather, the foregoing detailed description will provide those skilled in the art with a convenient road map for implementing the described embodiment or embodiments. It should be understood that various changes can be made in the function and arrangement of elements without departing from the scope defined by the claims, which includes known equivalents and foreseeable equivalents at the time of filing this patent application.

1. A patient care system for providing intervention courses to a patient having a physical health condition, said system comprising
a computing device which comprises an input interface and an output interface, and
a storage unit in which one or more application programs are stored, said one or more programs can be executed for providing instructions for a plurality of steps comprising
retrieving a list of a plurality of health conditions caused by said physical health condition;
providing the list of said plurality of health conditions through the output interface of the computing device;
in response to receiving a first input through the input interface of the computing device, that specifies one health condition of said plurality of health conditions, providing a group of questions corresponding to said one health condition through the output interface of the computing device; and
in response to receiving a second input through the input interface of the computing device, that comprises feedback to said group of questions, selecting one or more intervention courses based on said feedback to said group of questions, and providing said one or more intervention courses through the output interface of the computing device.

2. The system according to the claim 1, wherein said plurality of steps further comprising
in response to receiving an input through the input interface of the computing device, that specifies one or more health conditions of said plurality of health conditions, providing an output through the output interface of the computing device, that requires that said one health condition of said one or more health conditions is specified; and
and receiving said first input through the input interface of the computing device.

3. The system according to the claim 1, wherein the list of said plurality of health conditions comprises mental health conditions, dietary related conditions and/or physical exercise related conditions.

4. The system according to the claim 1, wherein the list of said plurality of health conditions comprises fatigue, insomnia, frustration, anxiety, and/or pain.

5. The system according to the claim 1, wherein said group of questions is designed to evaluate a scale corresponding to said one health condition, and said one or more intervention courses are provided depending on a scale score calculated based on the feedback to the group of questions.

6. The system according to the claim 5, wherein said one or more intervention courses can be classified into different levels comprising a level of relaxation tools and a level of cognitive behavioral treatments depending on the severity of said one health condition indicated by the calculated score.

7. The system according to the claim 1, wherein said input interface and said output interface comprise a same display and a same graphical user interface.

8. The system according to the claim 1, wherein said system further comprises a communication interface which could connect the computing device to a network or a Cloud.

9. The system according to the claim 8, wherein said application programs can be stored in the storage unit of said network or said Cloud.

10. The system according to the claim 8, wherein said one or more application programs are installed on a third party platform.

11. The system according to the claim 10, wherein said one or more application programs are WeChat mini-programs.

12. The system according to the claim 1, wherein said one or more application programs are mobile applications installed under iOS or Android environments.

13. The system according to the claim 1, wherein said computing device is a smart phone, a computer tablet, or a PC.

14. The system according to the claim 1, wherein the physical health condition is cancer, asthma, atopic dermatitis, chronic obstructive pulmonary disease, bullous pemphigoid or chronic spontaneous urticaria.

15. A method for providing intervention courses to a patient having a physical health condition, said method comprising
providing the computing system according to the claim 1, and
executing said one or more programs for providing instructions for said plurality of steps.
